(19) United States
(12) Patent Application Publication
(10) Pub. No.: US 2022/0087731 A1
(43) Pub. Date: Mar. 24, 2022

(54) REMOVABLE TIP FOR USE WITH ELECTROSURGICAL DEVICES

(71) Applicant: Jamison Alexander, Denison, TX (US)

(72) Inventor: Jamison Alexander, Denison, TX (US)

(21) Appl. No.: 17/533,662

(22) Filed: Nov. 23, 2021

Related U.S. Application Data
(63) Continuation-in-part of application No. 16/598,860, filed on Oct. 10, 2019, now Pat. No. 11,191,586.
Application No. 16/598,860 is a continuation-in-part of application No. 16/460,048, filed on Jul. 2, 2019, now Pat. No. 11,172,979.

(51) Int. Cl. A61B 18/14

(52) U.S. Cl. CPC A61B 18/1442; A61B 2018/00595; A61B 2218/008

(57) ABSTRACT

Disclosed herein are various embodiments of electrosurgical devices. In one embodiment a removable tip for use with an electrosurgical tool comprises a first tip member and a second tip member, each having a proximal end, a distal end, and a separating portion located therebetween; a smoke collector comprising at least one extension tube sealingly attached to a manifold; and an insertion member for inserting the removable tip within an opening in the electrosurgical tool. The extension tube(s) are affixed to an edge of the first or second separating portions. The extension tube(s) have a first opening at a first end closest to distal ends of the first and second tip members and a second end opposite the first end and are configured to allow smoke to be evacuated away from an area at the distal ends of the first and second tip members through the extension tube(s) into the manifold.

### CROSS-REFERENCE TO RELATED APPLICATION

This application is a continuation-in-part of U.S. application Ser. No. 16/598,860 (now U.S. Pat. No. 11,191,586), filed by Jamison Alexander on Oct. 10, 2019, which is a continuation-in-part of U.S. application Ser. No. 16/460,048 (now U.S. Pat. No. 11,172,979), filed by Jamison Alexander on Jul. 2, 2019, entitled “REMOVABLE TIP FOR USE WITH ELECTROSURGICAL DEVICES,” commonly assigned with this application and incorporated herein by reference in its entirety.

### TECHNICAL FIELD

This application is directed, in general, to surgical tools and, more specifically, to a removable tip which may be used with an electrosurgical tool for, among other things, cutting, clamping, and cauterizing tissue and vessels.

### BACKGROUND

Electrosurgical devices use high-frequency electrical current to cut, coagulate, and cauterize tissue and vessels and are used in a wide array of surgical procedures. Electrosurgical devices are generally handheld devices guided by a surgeon, making it possible to apply a high-frequency energy to tissue. Electrosurgical devices may be used, for example, to cut tissue, or in other examples, to seal or cauterize tissue and vessels. The energy level may be adjusted by the surgeon by means of control switches disposed in some embodiments on the handheld device, such as, e.g. one or more buttons.

During an operation, the surgeon generally has to navigate through several layers of tissue and vessels and may need to cut some tissue or vessels, while sealing or cauterizing other tissue or vessels. For example, electrosurgical devices are commonly used during surgery in order to stop bleeding by using an alternating current directly to heat tissue and thereby reduce blood loss and/or improve surgical vision. In some procedures, there may be a limited amount of space available for surgical instruments. In some embodiments, additional forceps or instruments may be necessary to clamp tissue or clamp around a vessel in order to use an electrosurgical device thereon or navigate around the tissue or vessel due to its size and/or location. In some cases, the surgeon may need additional space within the surgical field or may require an additional hand or set of hands to maneuver the instruments. Such interruptions in the operation may add additional time for the procedure and pose additional risks to the patient.

### BRIEF DESCRIPTION OF THE DRAWINGS

Reference is now made to the following descriptions taken in conjunction with the accompanying drawings, in which:

FIG. 1 is a perspective view of one embodiment of a removable tip for use with an electrosurgical device according to the present disclosure;

FIG. 2A is a perspective view of one embodiment of an electrosurgical tool according to the present disclosure, shown with a removable tip in a closed configuration;

FIG. 2B is a perspective view of the electrosurgical tool of FIG. 2A shown with the removable tip in a starting to open configuration;

FIG. 2C is a perspective view of the electrosurgical tool of FIG. 2A shown with the removable tip in a partially open configuration;

FIG. 2D is a perspective view of the electrosurgical tool of FIG. 2A shown with the removable tip in a fully open configuration;

FIG. 3 is a partial sectional view of another embodiment of a removable tip for use with an electrosurgical device according to the present disclosure;

FIG. 4A is a perspective view of one embodiment of an electrosurgical tool according to the present disclosure, shown with a removable tip in a closed configuration;

FIG. 4B is a perspective view of the electrosurgical tool of FIG. 4A shown with the removable tip in a partially open configuration;

FIG. 4C is a perspective view of the electrosurgical tool of FIG. 4A shown with the removable tip in an open configuration;

FIG. 5A is a perspective view of another embodiment of a removable tip for use with an electrosurgical device according to the present disclosure, shown with the removable tip in a closed configuration;

FIG. 5B is a perspective view of the removable tip shown in FIG. 5A, shown with the removable tip in an open configuration;

FIG. 6A is a perspective view of yet another embodiment of a removable tip for use with an electrosurgical device according to the present disclosure, shown with the removable tip in a closed configuration;

FIG. 6B is a perspective view of the removable tip shown in FIG. 6A, shown with the removable tip in an open configuration;

FIG. 6C is an environmental view of the removable tip shown in FIGS. 6A and 6B, shown with the removable tip in an open configuration;

FIG. 7A is a perspective view of another embodiment of a removable tip for use with an electrosurgical device according to the present disclosure, shown with the removable tip in an open configuration;

FIG. 7B is a perspective view of the removable tip shown in FIG. 7A, shown with the removable tip in a closed configuration;

FIG. 7C is an environmental view of the removable tip shown in FIGS. 7A and 7B, shown with the removable tip in a closed configuration;

FIG. 8A is a perspective view of an embodiment of a smoke collector for use with a removable tip according to the present disclosure;

FIG. 8B is a perspective view of an embodiment of a handheld electrosurgical device with a shroud according to the present disclosure;

FIG. 9A is a perspective view of another embodiment of a removable tip including a smoke collector for use with an electrosurgical device according to the present disclosure, shown with the removable tip in an closed position;

FIG. 9B is a perspective view of the removable tip including the smoke collector shown in FIG. 9A, shown with the removable tip in a open position; and

FIG. 9C is an environmental view of the removable tip including the smoke collector shown in FIGS. 9A and 9B, shown with the removable tip in a open position.

### DETAILED DESCRIPTION

Two primary types of electrosurgical device are known, namely bipolar and monopolar devices. In monopolar arrangements the electrosurgical device is provided with an active electrode and a return electrode is attached to the patient. The electric current flows from the active electrode into the body and returns through the return electrode (which is connected to a grounding circuit). The current density decreases rapidly with distance away from the electrode such that the heating of tissue is localized to the tip of the electrosurgical device. In bipolar devices, a pair of electrodes, for example the tips of forceps, are each connected to the supply circuit and no return electrode is required. When tissue is engaged by or proximal to the pair of electrodes, the high frequency electric current flows through the device and tissue providing a localized heating of the tissue.

Conventional electrosurgical devices may include a single member tip or may include a forceps design which is substantially based upon traditional surgical forceps. As such, a surgeon may have to change electrosurgical instruments, from a single tip to a forceps type design, depending on the function required during a procedure. As such, switching between conventional tips and forceps may add time and risk to a patient during a surgical procedure. In addition, conventional electrosurgical devices are not adjustable to adapt to various body cavity areas or depths and also have the ability to cut, clamp, and cauterize both tissue and vessels.

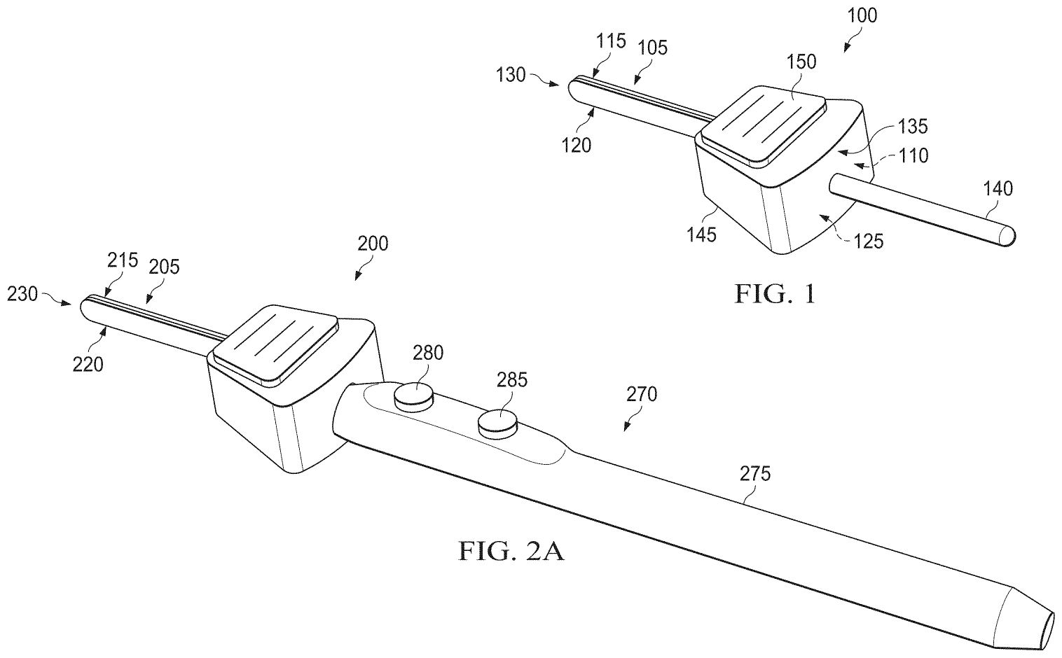

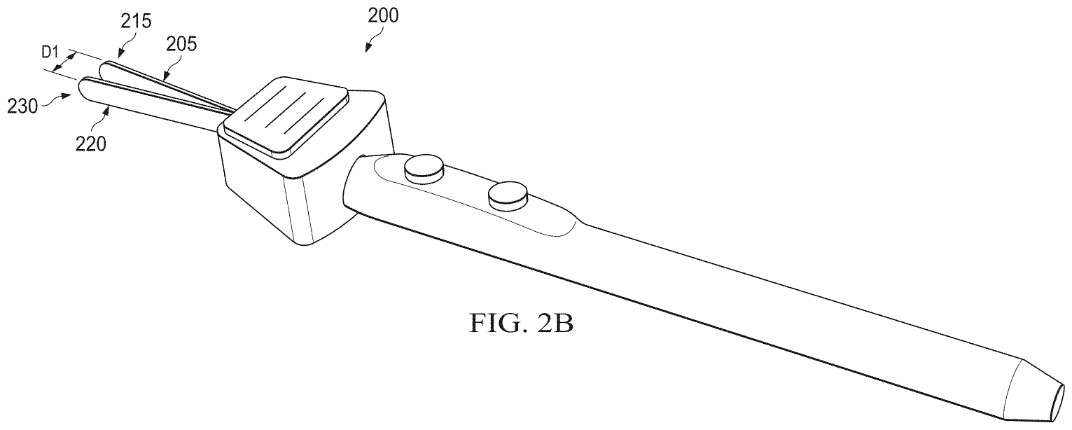

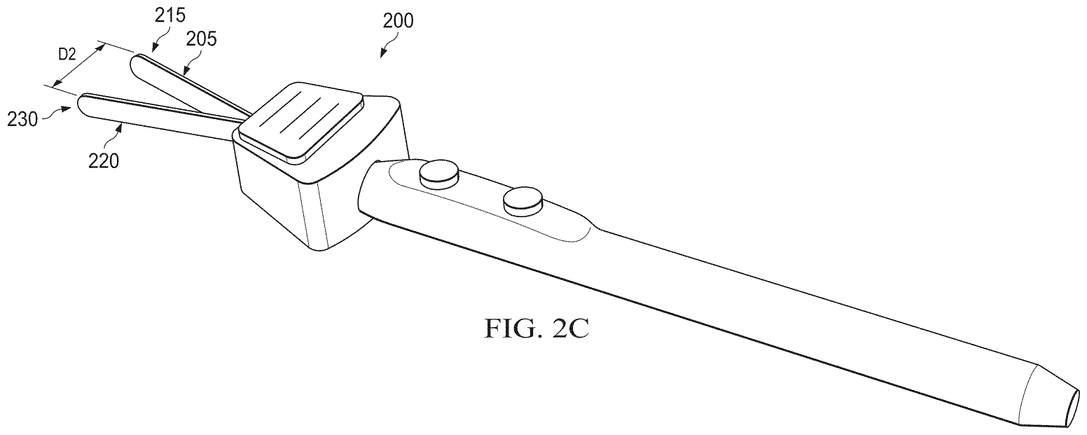

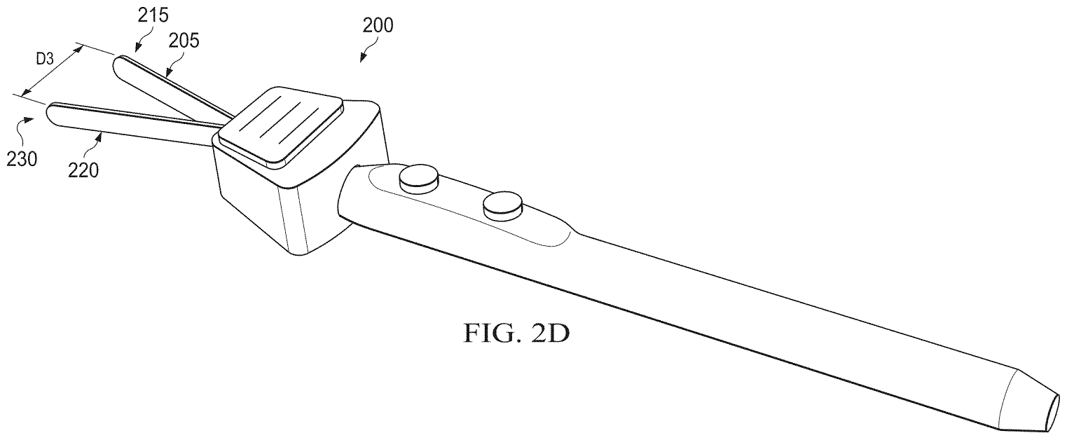

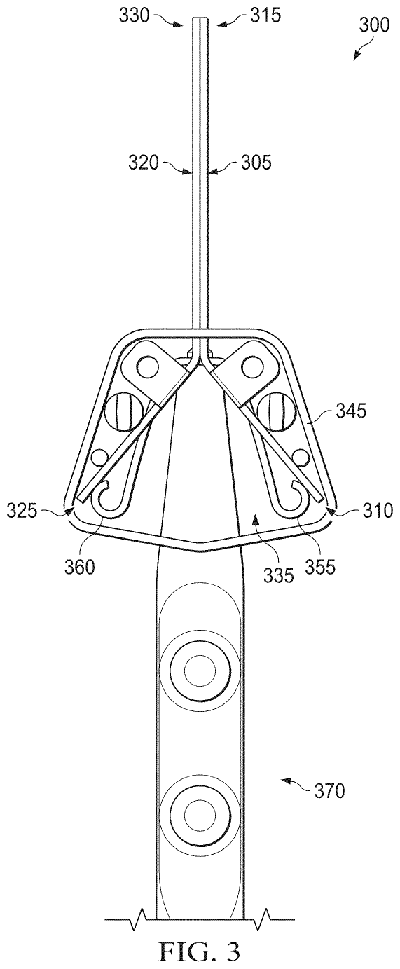

Disclosed herein are embodiments of a removable tip for use with an electrosurgical tool which may be used in surgical procedures. Referring to the drawings, and more specifically, FIG. 1, there is shown a removable tip 100 representing one embodiment of the present disclosure. The removable tip 100 comprises a first tip member 105 having a proximal end 110 and a distal end 115. A second tip member 120 likewise has a proximal end 125 and a distal end 130. In some embodiments, at least one or both of the first tip member 105 and the second tip member 120 may include at least one electrically conductive surface near the distal ends 115 and 130 thereof. The first tip member 105 and second tip member 120 may be configured in some embodiments to move from a closed position (as shown in FIG. 1 and FIG. 2A) where the distal end 115 of the first tip member 105 and the distal end 130 of the second tip 120 member are proximate one another, and an open position where the distal end 115 of the first tip member 105 and the distal end 130 of the second tip member 120 are separated by a distance from one another. (Varying stages of open positions will be illustrated in FIGS. 2B-2D). In some embodiments, an actuator 135 may be coupled to the first tip member 105 and the second tip member 120 for moving the first and second tip members 105 and 120 between the closed position and one or more open positions.

In some embodiments, an insertion member 140 may be coupled with the proximal ends 110 and 125 of the first and second tip members 105 and 120, the insertion member 140 for inserting the removable tip 100 within an opening in an electrosurgical tool. In some embodiments, the insertion member 140 may be rotatable within the opening in the electrosurgical tool such that the removable tip 100 may be adjusted to varying angles with respect to the electrosurgical tool. By enabling adjustment of an angle of the removable tip, a surgeon may be able to better maneuver the removable tip 100 within a body cavity than conventional electrosurgical devices. In some examples, the removable tip 100 may need to be rotated to achieve a better placement in the body cavity while enabling the surgeon's hand to be placed in a better or more comfortable or maneuverable position.

In some embodiments, the removable tip 100 may include a housing 145 positioned between the distal ends 115 and 130 and the proximal ends 110 and 125 of the first and second tip members 105 and 120. In some embodiments, the actuator 135 may be positioned at least partially within the housing 145. In other embodiments, the housing 145 may be positioned at least partially over the insertion member 140.

In some embodiments, the actuator 135 may include a switch 150. The switch 150, in embodiments not including a housing 145, may be located in various locations on or about the actuator 135. The switch 150 may be configured to move linearly upon pressure applied by a finger or thumb and move the first and second tip members 105 and 120 between closed and open positions, and various positions in between.

In some embodiments, the actuator 135 may include one or more separating members, which may be positioned, in some embodiments within the housing 145. The one or more separating members may be positioned about or between the first and second tip members 105 and 120 for separating the distal ends 115 and 130 of the first and second tip members 105 and 120 when the surgeon engages the actuator 135, which in some embodiments, may be operation of the switch 150. The separating members may also act to move the distal ends 115 and 130 of the first and second tip members 105 and 120 closer together when the actuator 135 may be engaged again, in some embodiments, moving the switch 150 in a second direction.

The removable tip 100 may be adjusted between a closed position and varying open positions (examples shown in FIGS. 2A-2D) depending on what the surgeon may need to use the removable tip 100 for during a surgical procedure. In some examples, the removable tip 100 may need to be in a closed position, for example, when cutting tissue may be needed. However, in certain procedures, clamping the distal end 115 of the first tip member 105 and the distal end 130 of the second tip 120 about a vessel may be useful rather than using a second instrument to clamp around the vessel before applying current from a conventional electrosurgical tip, for example, for cutting or cauterizing a vessel.

The removable tip 100 may also accommodate varying lengths, both longer and shorter, of first tip members 105 and second tip members 120 such that the removable tip 100 may reach deeper within some body cavities without changing the entire electro surgical tool, such as previously required with conventional fixed electrosurgical tools. In some embodiments, the first tip member 105 and second tip members 120 may be adjustable and/or extendable to varying lengths. In other embodiments, the insertion member 140 may also be adjustable and/or extended to varying lengths.

Referring now to FIGS. 2A through 2D illustrate another embodiment of a removable tip 200 shown coupled into an electrosurgical tool 270, shown in various stages. FIG. 2A illustrates the removable tip in a closed position, wherein distal ends 215 and 230 of first and second tip members 205 and 220 are proximate one another.

FIGS. 2B through 2D illustrate the removable tip 200 in various open stages, wherein the distal ends 215 and 230 of the first and second tip members 205 and 220 are separated by various distances. FIG. 2B illustrates the removable tip 200 as the first and second tip members 205 and 220 are beginning to open and the distal ends 215 and 230 of the first and second tip members 205 and 220 begin to separate by a first distance D1. FIG. 2C illustrates the removable tip 200 in where the distal ends 215 and 230 first and second tip members 205 and 220 are separate by a second distance D2, which may be about halfway open, wherein halfway open is about 45-55% of fully open. FIG. 2D illustrates the removable tip 200 in a fully open position, where the distal ends 215 and 230 of first and second tip members 205 and 220 are separate by a third distance D3. Fully open, in this disclosure means the distal ends 215 and 230 of the first and second tip members 205 and 220 are within 95-100% open to the maximum functional separation of the distal ends 215 and 230 of the first and second tip members 205 and 220.

Referring again to FIG. 2A, the electrosurgical tool 270 includes a handheld electrosurgical unit 275. The handheld electrosurgical unit 275 may connect with a wall unit, in some embodiments via a plug-in connection such as a wire or cord. The wall unit may supply the electrical current to the electrosurgical tool 270. The first tip member 205 and second tip member 220 may include one or more conductive surfaces for conducting current to tissue to which it may be applied or clasped about. The handheld electrosurgical unit 275 may include, in some embodiments, at least a first button 280 to activate a cutting mode and a second button 285 to activate a cauterizing mode. Additional buttons or functions may be included in some embodiments of the handheld electrosurgical unit 275. Further, some embodiments of the removable tip 200 may be used in monopolar mode while others may be used in bipolar mode.

Referring now to FIG. 3, there is shown another embodiment of a removable tip 300 coupled into an electrosurgical tool 370 according to principles of the disclosure. The removable tip 300 may include a first tip member 305 having a proximal end 310 and a distal end 315. A second tip member 320 likewise has a proximal end 325 and a distal end 330. In some embodiments, at least one or both of the first tip member 305 and the second tip member 320 may include at least one electrically conductive surface near the distal ends 315 and 330 thereof. The first tip member 305 and second tip member 320 may be configured in some embodiments to move from a closed position to an open position, as discussed hereinabove and shown in FIG. 1 through FIG. 2D.

In some embodiments, an actuator 335 may be coupled to the first tip member 305 and the second tip member 320 for moving the first and second tip members 305 and 320 between the closed position and one or more open positions. The actuator 335 may, in some embodiments, be positioned within a housing 345. The view of FIG. 3 is a partial section-view, showing the inside of housing 345.

In some embodiments, the actuator 335 may include a first separating member 355 and a second separating member 360. In this embodiment, the first separating member 355 is adjacent the proximal end 310 of the first tip member 305 and the second separating member 360 is positioned adjacent the proximal end 325 of the second tip member 320, but the first separating member 355 and second separating member 360 may be positioned between the first and second tip members 305 and 320 at any location that enables the actuator to move the removable tip 300 between closed position and an open position. In another embodiment, the first separating member 355 and the second separating member 360 may be positioned at or incorporating into opposing ends of a single separating component.

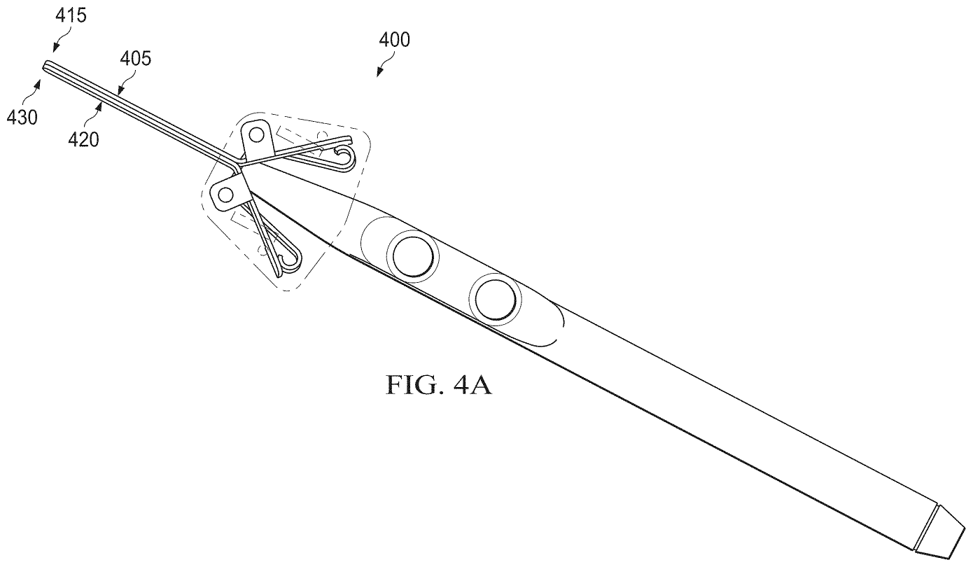

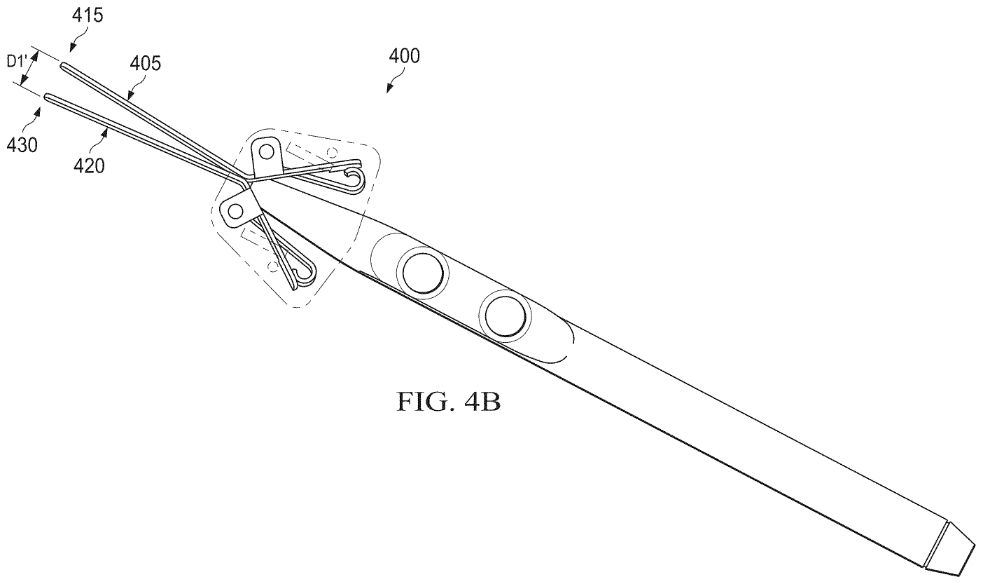

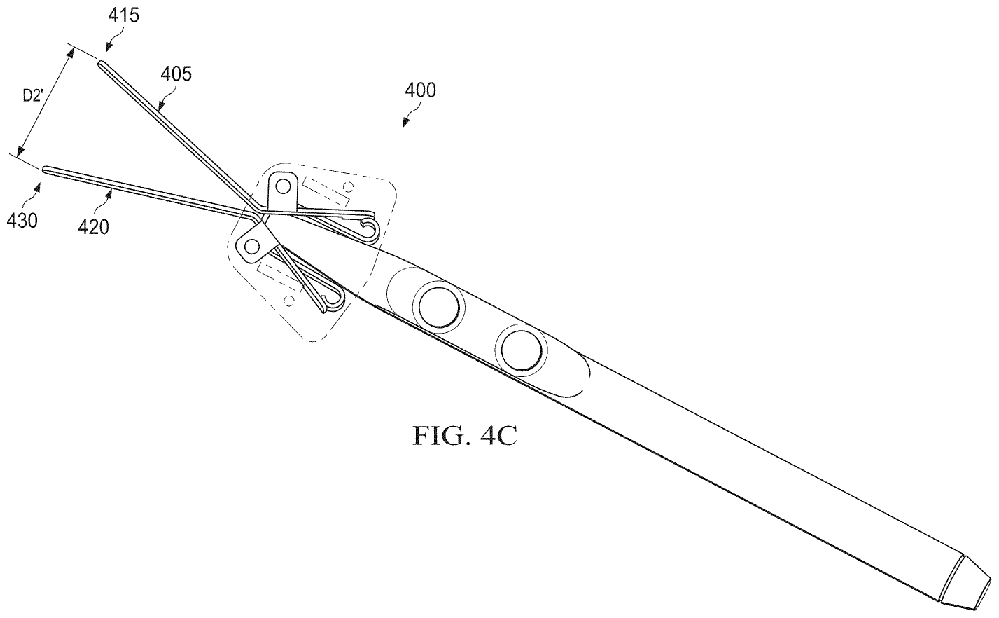

Referring now to FIGS. 4A-4C, there is shown another embodiment of a removable tip 400 shown in 3 different positions. FIG. 4A shows the removable tip 400 in a closed position, wherein a distal end 415 of a first tip member 405 is proximate a distal end 430 of a second tip member 420. FIG. 4B illustrates the removable tip 400 in an opening position, wherein the distal ends 415 and 430 are separate by a distance D1′. FIG. 4C illustrates the removable tip 400 in an open position, wherein the distal ends 415 and 430 are separate by a distance D2′.

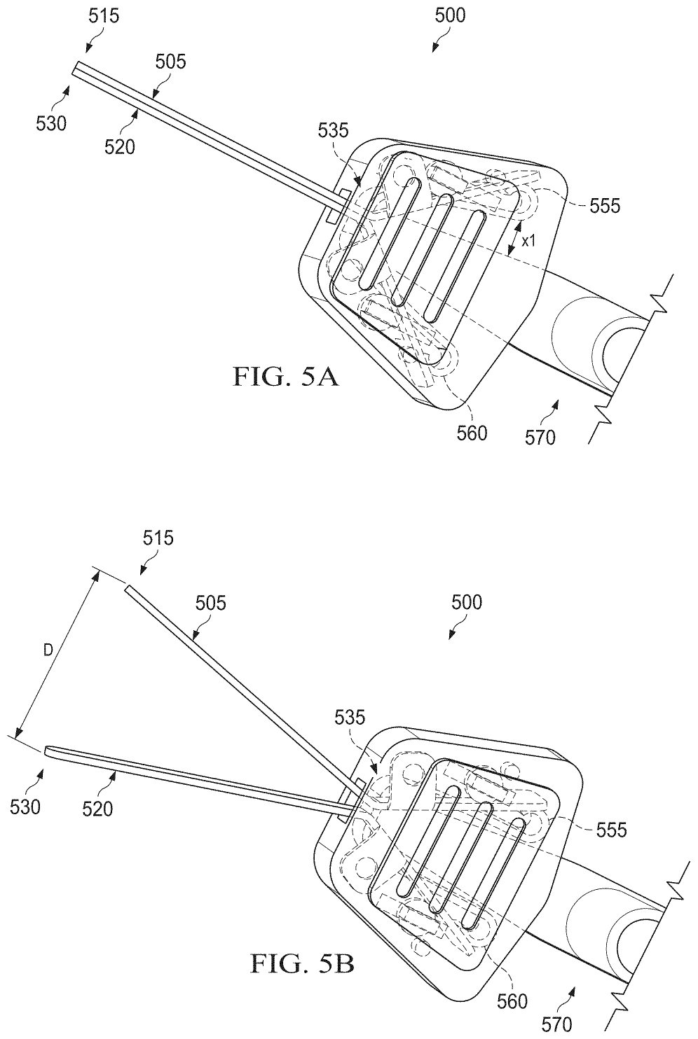

Referring now to FIGS. 5A and 5B there is shown another embodiment of a removable tip 500 shown in a closed position in FIG. 5A and an open position in FIG. 5B. As shown in FIG. 5A, in this embodiment, when the removable tip 500 is closed, a first separating member 555 and a second separating member 560 of an actuator 535 are each positioned at a distance xl away from a distal end of an electrosurgical tool 570 onto which the removable tip 500 may be coupled. When the removable tip 500 is in an open position, the first and second separating members 555 and 560 may be proximate the distal end of the electrosurgical tool 570 such that there is little or no distance between the first and second separating members 555 and 560 and the distal end of the electrosurgical tool 570. When the removable tip 500 is in varying open positions, similar to those illustrated in FIG. 2B-2D, the distance between the first and second separating members 555 and 560 and the distal end of the electrosurgical tool 570 may vary accordingly.

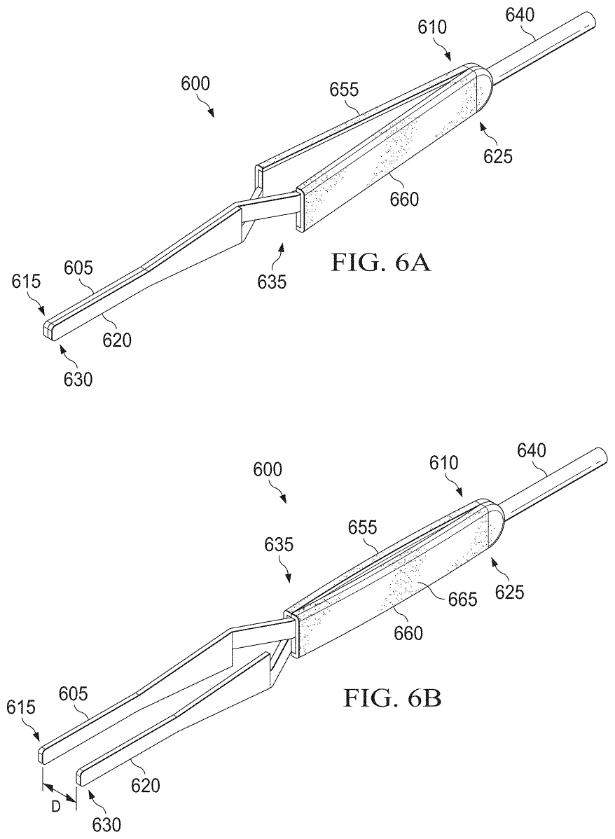

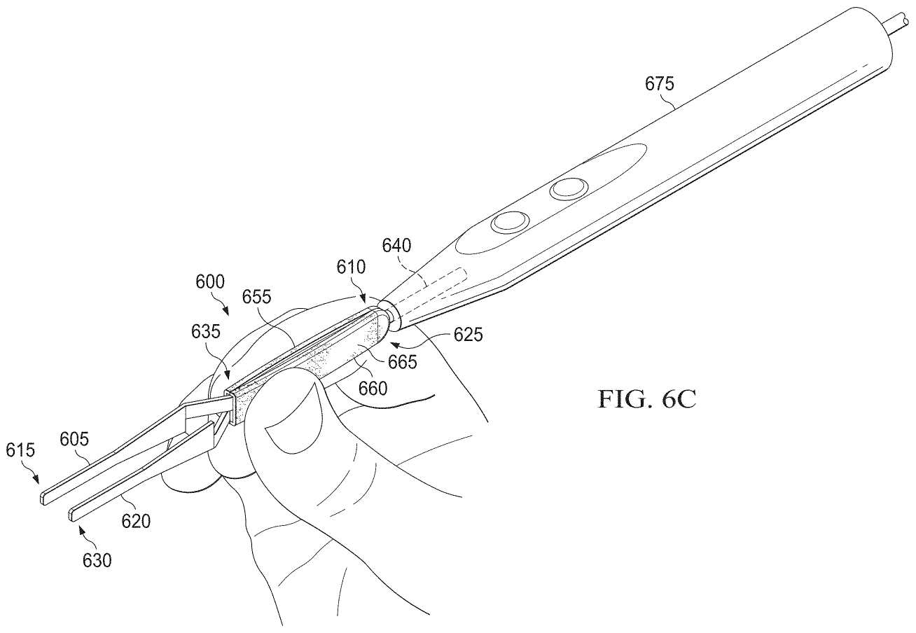

Referring now to FIGS. 6A-6C, there is shown yet another embodiment of a removable tip 600 configured as cross-linking tweezers coupled into a handheld electrosurgical unit 675 according to principles of the disclosure. The removable tip 600 may include a first tip member 605 having a proximal end 610 and a distal end 615. A second tip member 620 likewise has a proximal end 625 and a distal end 630. In some embodiments, at least one or both of the first tip member 605 and the second tip member 620 may include at least one electrically conductive surface at or near the distal ends 615 and 630 thereof. The first tip member 605 and second tip member 620 may be configured in some embodiments to move from a closed position, as shown in FIG. 6A to an open position, as shown in FIG. 6B.

In some embodiments, an insertion member 640 may be coupled with the proximal ends 610 and 625 of the first and second tip members 605 and 620, the insertion member 640 for inserting the removable tip 600 within an opening in the handheld electro surgical unit 675. In some embodiments, the insertion member 640 may be rotatable within the opening in the electrosurgical unit 675 such that the removable tip 600 may be adjusted to varying angles with respect to the electrosurgical tool.

In some embodiments, an actuator 635 may be coupled to the first tip member 605 and the second tip member 620 for moving the first and second tip members 605 and 620 between the closed position and one or more open positions. In this embodiment, the actuator 635 comprises a portion of the first and second tip members 605 and 620.

In some embodiments, the actuator 635 may include a first separating member 655 and a second separating member 660. In one embodiment, the first separating member 655 and the second separating member 660 each comprise a portion of the first and second tip members 605 and 620. In some embodiments, to actuate the first and second tip members 605 and 610 from a closed position as shown in FIG. 6A to an open position shown in FIG. 6B, axial pressure may be applied to the first separating member 655 and the second separating member 660, moving the first separating member 655 and the second separating member 660 toward each other as the distal ends 615 and 630 move apart from one another. In some examples, the pressure may be applied to at least exterior surfaces of the first and second separating members 655 and 660.

In some embodiments, at least a portion of the first and second tip members 605 and 620 may be removable and/or replaceable. In one embodiment, the distal ends 615 and 630 may be removed at or above the first separating member 655 and the second separating member 660. The distal ends 605 and 620 may couple with the first separating member 655 and the second separating member 660 by a snap fit, tension fit, a locking fit, and other traditional coupling means for removing and replacing a distal end of a longitudinal member such as the first and second tip members 605 and 620.

Referring to FIG. 6C, there is shown an environmental view of the removable tip 600, shown with the actuator 635 engaged such that the distal ends 615 and 630 of the first and second tip members 605 and 620 are opened apart from each other. In this embodiment, the actuator 635 is shown engaged by fingers applying axial pressure to the first and second separating members 655 and 660. In one embodiment, the first separating member 655 and second separating member 660 may include a coating or covering 665 on at least an exterior surface of each, but in some embodiments, the coating or covering may cover or coat most or all surfaces of the first and second separating members 655 and 660. The coating 665 may comprise polymers, rubbers, plastics, and any other suitable material which may provide, in some embodiments, insulation from any electrical current running through the removable tip 600 and may also, in other embodiments, provide a non-slip grip, and/or comfort grip to a surgeon handling the removable tip 600.

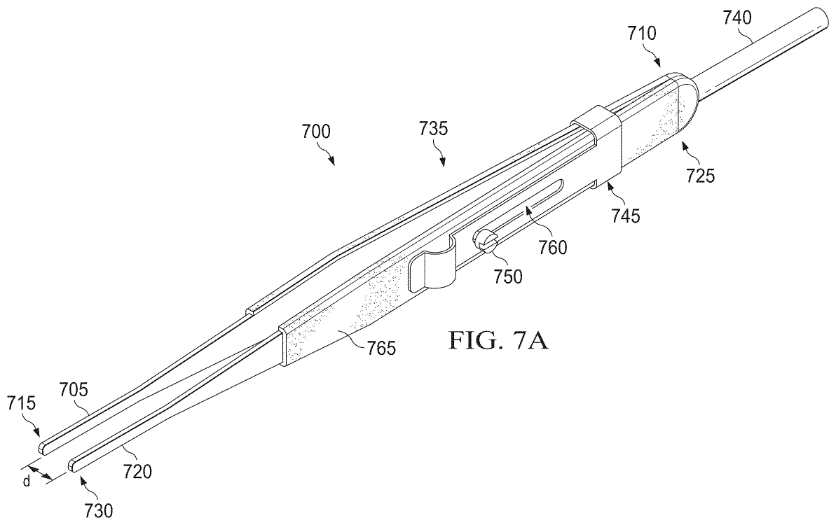

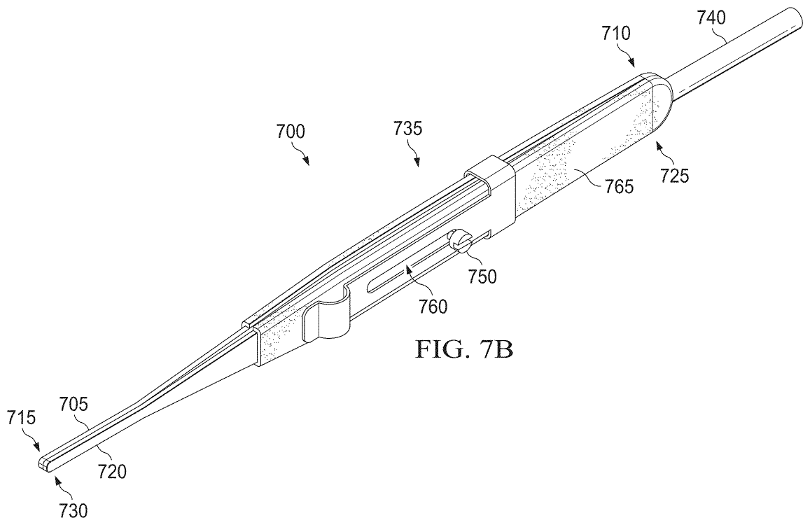

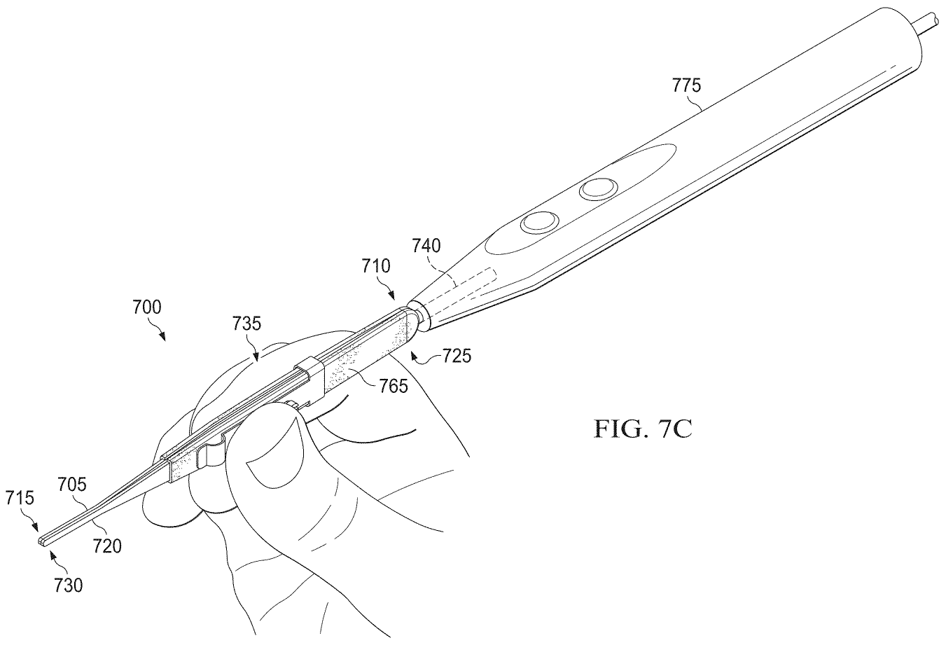

Referring now to FIGS. 7A-7C, there is shown yet another embodiment of a removable tip 700 configured as standard tweezers coupled into a handheld electrosurgical unit 775 according to principles of the disclosure. The removable tip 700 may include a first tip member 705 having a proximal end 710 and a distal end 715. A second tip member 720 likewise has a proximal end 725 and a distal end 730. In some embodiments, at least one or both of the first tip member 705 and the second tip member 720 may include at least one electrically conductive surface near the distal ends 715 and 730 thereof. The first tip member 705 and second tip member 720 may be configured in some embodiments to move from a closed position to an open position, as discussed hereinabove and shown in FIG. 7A-7C. An insertion member 740 may extend from the removable tip near the proximal ends 710 and 725, the insertion member 740 for inserting the removable tip 700 within an opening in the handheld electrosurgical unit 775.

In some embodiments, the removable tip may include an actuator 735 for moving the distal ends 715 and 730 of the first and second tip members 705 and 720 between a closed position and one or more open positions. In one embodiment, the actuator 735 may comprise part of the first tip member 705 and the second tip member 720. In one embodiment, the actuator 735 may include a locking mechanism 745, which in one embodiment includes a locking pin 750 positioned within a lateral slot 760. In some embodiments, the locking mechanism may be coupled around both the first and second tip members 705 and 720, and in some embodiments, near the proximal ends 710 and 725 thereof. In one embodiment, the first and second tip members 705 and 720 are in an “open position” as shown in FIG. 7A, which the distal ends 715 and 730 are separated by a distance d. To move the distal ends 715 and 730 toward a “closed position” a shown in FIG. 7B, axial pressure may be applied to the first and second tip members 705 and 720 and the locking mechanism 745 may be moved toward the distal ends 715 and 730. Once the distal ends 715 and 730 are closed together, in some embodiments, the pin 750 may be engaged to maintain the locking mechanism in a locked position. The pin 750 may be engaged by turning the pin 750 to a locked position, or in some embodiments may be locked in a compressed position.

Referring to FIG. 7C, there is shown an environmental view of the removable tip 700, shown with the actuator 735 engaged such that the distal ends 715 and 730 of the first and second tip members 705 and 720 are closed proximal each other. In this example, the actuator may include a coating or covering 765 at least on an outer surface. The coating 765 may comprise polymers, rubbers, plastics, and any other suitable material which may provide, in some embodiments, insulation from any electrical current running through the removable tip 700 and may also, in other embodiments, provide a non-slip grip, and/or comfort grip to a surgeon handling the removable tip 700.

In some embodiments, all of part of the removable tip 700 may include a non-stick coating which may be placed, in some embodiments, on at least the distal ends 715 and 730 of the first and second tip members 705 and 720 to reduce build-up of charred tissue thereon and also reduce a risk of thermal injuries. Several different types of non-stick coatings have been used or suggested for application to electrosurgical electrodes. Some of the different non-stick coatings or materials include fluorinated hydrocarbon materials, polytetrafluoroethylene (PTFE), perfluoroalkoxy, flexible silicone elastomers, ceramic composites, paralyene siloxane polymers, epoxy modified rigid silicone powder, silicone resin, and other suitable non-stick coatings.

During an operation when a handheld electrosurgical device is used, often smoke is generated as tissue is either cauterized or cut with the electrosurgical device. This smoke can cause many different problems for the surgeon performing the operation with the electrosurgical device. For example, the smoke can occlude a view of the tissue being operated on, making precision cauterization or cutting difficult. Additionally, the smoke may be carcinogenic, potentially harming those involved with the operation. Thus, it is advantageous to evacuate the smoke to improve the view of the tissue being operated on and to remove any potentially dangerous substances during the operation.

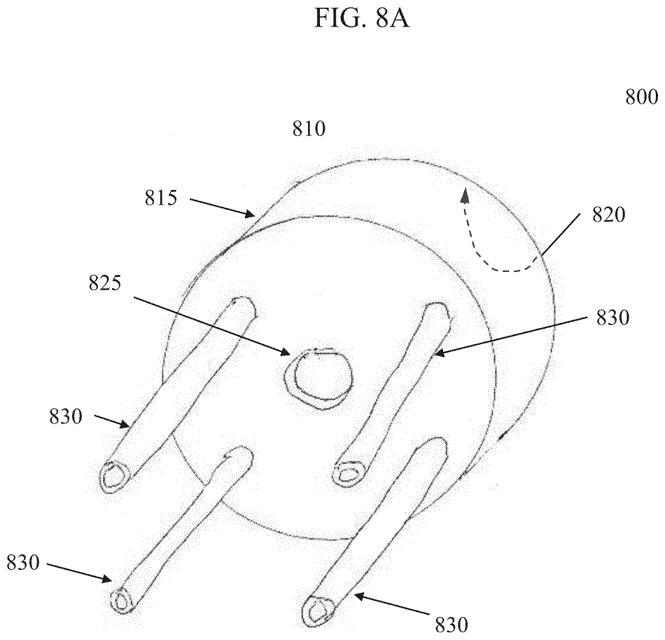

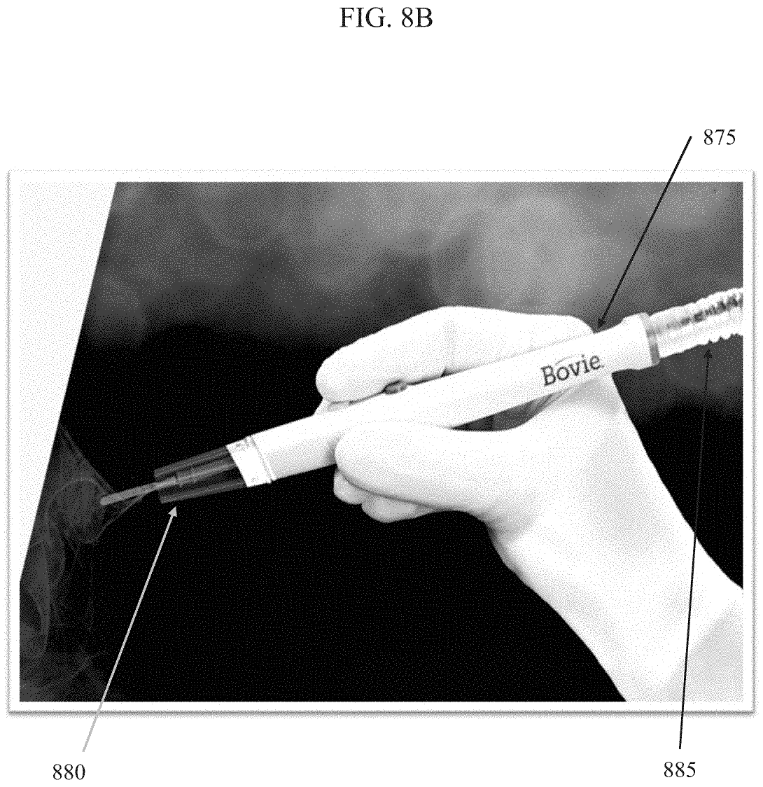

Referring now to FIG. 8A, there is shown an embodiment of a smoke collector 800 used to evacuate smoke while using a handheld electrosurgical device in conjunction with a removable tip. Smoke collector 800 includes a manifold 810. Manifold 810 has an exterior surface 815 and an interior surface 820. Manifold 810 also includes an opening 825. Opening 825 allows an insertion member of a removable tip (e.g., insertion member 640 or 740 as disclosed above) for inserting the removable tip (e.g., removable tip 600 or 700 as disclosed above) within an opening of a handheld electrosurgical unit (e.g., handheld electrosurgical units 675 or 775 as disclosed above). Smoke collector 800 also includes at least one extension tube 830 sealingly attached to manifold 810. In the example embodiment depicted in FIG. 8A, four extension tubes 830 are shown. It is within the scope of this disclosure that smoke detector 800 can include any number of extension tubes 830. Smoke collector 800 can be made of any pliable thermoplastic material, for example medical grade silicone with a thickness that ranges from about 0.05 to 0.40 millimeters. FIG. 8B shows an embodiment of handheld electrosurgical unit 875. Handheld electrosurgical unit 875 is similar to handheld electrosurgical units 675 and 775 as disclosed above, however handheld electrosurgical unit 875 further includes shroud 880. Shroud 880 is made of a similar thermoplastic material as used for smoke collector 800. Handheld electrosurgical unit 875 also includes a sealed connection tube 885 at its proximal end which is connected to a vacuum system (not shown) for the evacuation of smoke through and away from handheld electrosurgical unit 875 and a removable tip.

It is important that there is a tight fit between manifold 810 of smoke collector 800 and shroud 880 of handheld electrosurgical unit 875 so that the smoke can be evacuated from an area of the removable tip and cannot escape. This fit can be provided in many ways. In one example, inner surface 820 of manifold 810 of smoke collector 800 tightly fits over an outer surface of shroud 880. In another example, outer surface 815 of manifold 810 tightly fits into an inner surface of shroud 880. In yet another example, multiple sizes of manifold 810 can be provided so that one of the multiple sizes can facilitate a tight fit between either inner surface 820 of manifold 810 and the outer surface of shroud 880 or outer surface 815 of manifold 810 and the inner surface of shroud 880.

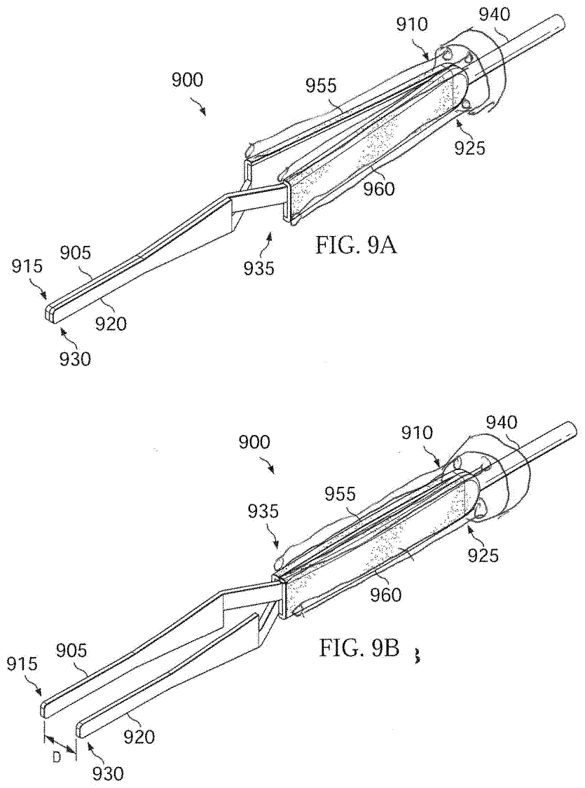

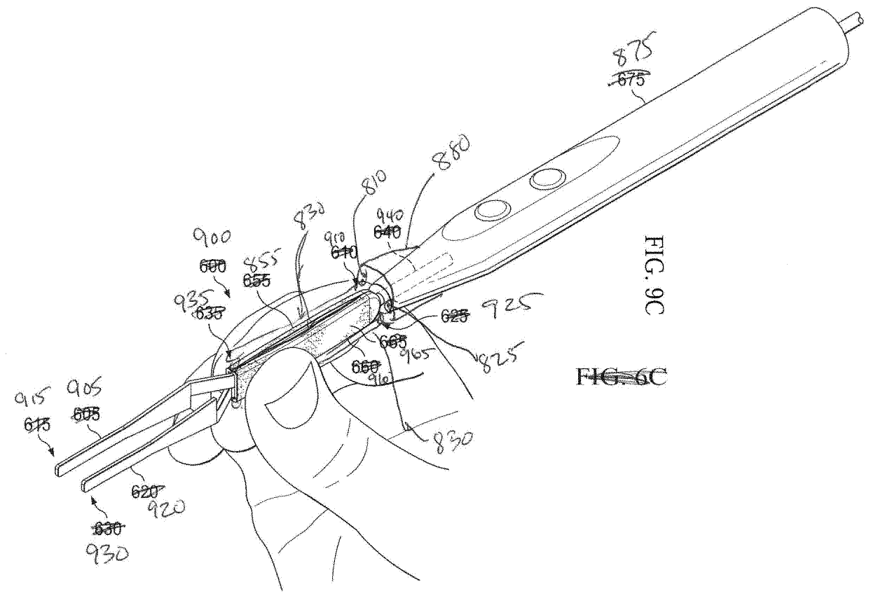

Referring now to FIGS. 9A-9C, there is shown yet another embodiment of a removable tip 900 coupled into a handheld electrosurgical unit, for example handheld electrosurgical unit 875 of FIG. 8B, according to principles of the disclosure. Removeable tip 900 is configured as cross-linking tweezers similar to the configuration of FIGS. 6A-6C. However, removable tip 900 could also be configured as standard tweezers as depicted in FIGS. 7A-7C or any other tweezer configuration. Handheld electrosurgical device 875 of FIGS. 9A-9C is similar to handheld electrosurgical device 875 of FIG. 8B and includes shroud 880 and sealed connection tube 885. The removable tip 900 includes smoke collector 800 as depicted in FIG. 8A and described above. In the embodiment of FIGS. 9A-9C, the inner surface of shroud 880 fits over outer surface 815 of manifold 810 of smoke collector 800. As discussed above, the outer surface of shroud 880 fits inside inner surface 820 of manifold 810 of smoke collector 800 in a different embodiment and in yet another embodiment a different sized manifold 810 is used to facilitate a tight fit between either inner surface 820 of the different sized manifold 810 and the outer surface of shroud 880 or outer surface 815 of the different sized manifold 810 and the inner surface of shroud 880. Each of the at least one extension tubes 830 is attached to corresponding edges of first separating member 955 and/or second separating member 960 using conventional materials. Distal ends of the extension tubes 935 substantially align with corresponding distal ends of first separating member 955 and second separating member 960. This configuration allows for smoke to be evacuated from an area proximate the distal ends of first separating member 955 and second separating member 960, the smoke evacuated through extension tubes 830, manifold 810, shroud 880, handheld electrosurgical unit 875, and sealed connection tube 885 into the vacuum system (not shown) allowing for better visibility of the tissue being operated on with the handheld electrosurgical unit 875 and removing any potentially dangerous substances during the operation.

As with the removable tip 600 of FIGS. 6A-6C, removable tip 900 may include a first tip member 905 having a proximal end 910 and a distal end 915. A second tip member 920 likewise has a proximal end 925 and a distal end 930. In some embodiments, at least one or both of the first tip member 905 and the second tip member 920 may include at least one electrically conductive surface at or near the distal ends 915 and 930 thereof. The first tip member 905 and second tip member 920 may be configured in some embodiments to move from a closed position, as shown in FIG. 9A to an open position, as shown in FIG. 9B.

In some embodiments, an insertion member 940 may be coupled with the proximal ends 910 and 925 of the first and second tip members 905 and 920, the insertion member 940 for inserting the removable tip 900 within an opening in the handheld electro surgical unit 875. In some embodiments, the insertion member 940 may be rotatable within the opening in the electrosurgical unit 875 such that the removable tip 900 may be adjusted to varying angles with respect to the handheld electrosurgical tool.

In some embodiments, first tip member 910 may include a first separating member 955 and second tip member 925 may include a second separating member 960. In some embodiments, to move the first and second tip members 905 and 910 from a closed position as shown in FIG. 9A to an open position shown in FIG. 9B, axial pressure may be applied to the first separating member 955 and the second separating member 960, moving the first separating member 955 and the second separating member 960 toward each other as the distal ends 915 and 930 move apart from one another. In some examples, the pressure may be applied to at least exterior surfaces of the first and second separating members 955 and 960.

In some embodiments, at least a portion of the first and second tip members 905 and 920 may be removable and/or replaceable. In one embodiment, distal end 915 of first tip member 905 and distal end 930 of second tip member 920 may be removed at or distal to a distal end of first separating member 955 and/or second separating member 960. Distal ends 905 and 920 may couple with the first separating member 955 and the second separating member 960, respectively, by a snap fit, tension fit, a locking fit, and other traditional coupling means for removing and replacing a distal end of a longitudinal member such as first and second tip members 905 and 920.

Referring to FIG. 9C, there is shown an environmental view of the removable tip 900, shown such that the distal ends 915 and 930 of the first and second tip members 905 and 920 are opened apart from each other. In this embodiment, fingers apply axial pressure to the first and second separating members 955 and 960. In one embodiment, the first separating member 955 and second separating member 960 may include a coating or covering 965 on at least an exterior surface of each, but in some embodiments, the coating or covering may cover or coat most or all surfaces of the first and second separating members 955 and 960. The coating 965 may comprise polymers, rubbers, plastics, and any other suitable material which may provide, in some embodiments, insulation from any electrical current running through the removable tip 900 and may also, in other embodiments, provide a non-slip grip, and/or comfort grip to a surgeon handling the removable tip 900.

Aspects of electrosurgical devices disclosed herein include the following:

A: A removable tip for use with an electrosurgical tool comprising a first tip member having a proximal end, a distal end, and a first separating portion located therebetween; a second tip member having a proximal end, a distal end, and a second separating portion located therebetween, where the proximal end of the second tip member is directly coupled with the proximal end of the first tip member; a smoke collector comprising at least one extension tube and a manifold; and an insertion member directly coupled with the proximal ends of the first and second tip members, the insertion member for inserting the removable tip within an opening in the electrosurgical tool. The at least one extension tube is affixed to an edge of the first or second separating portions, the at least one extension tube having a first opening and a second opening, the first opening at a first end of the at least one extension tube closest to the distal ends of the first and second tip members and the second opening at a second end of the at least one extension tube opposite the first end of the at least one extension tube. Each of the second ends of the at least one extension tube is fitted into a corresponding opening of the manifold. The at least one extension tube is configured to allow smoke to be evacuated away from an area at the distal ends of the first and second tip members through the first end of the at least one extension tube into the manifold through the second end of the at least one extension tube.

B: An electrosurgical tool comprising a handheld electrosurgical unit and a removable tip. The handheld electrosurgical unit configured to be connected with an electrosurgical wall unit and including at least a first button to activate a cutting mode, a second button to activate a cauterizing mode, and a shroud configured to evacuate smoke through the electrosurgical tool. The removable tip comprising a first tip member having a proximal end, a distal end, and a first separating portion located therebetween; a second tip member having a proximal end, a distal end, and a second separating portion located therebetween, where the proximal end of the second tip member is directly coupled with the proximal end of the first tip member; a smoke collector comprising at least on extension tube and a manifold; and an insertion member directly coupled with the proximal ends of the first and second tip members, the insertion member for inserting the removable tip within an opening in the electrosurgical tool. The at least one extension tube is affixed to an edge of the first or second separating portions, the at least one extension tube having a first opening and a second opening, the first opening at a first end of the at least one extension tube closest to the distal ends of the first and second tip members and the second opening at a second end of the at least one extension tube opposite the first end of the at least one extension tube. Each of the second ends of the at least one extension tube is sealingly attached to the manifold. The at least one extension tube configured to allow smoke to be evacuated away from an area at the distal ends of the first and second tip members through the first end of the at least one extension tube into the manifold through the second end of the at least one extension tube to the shroud of the electrosurgical device.

Aspects A and B may have one or more of the following additional elements in combination:

Element 1: where the removable tip is configured as cross-linking tweezers.

Element 2: wherein when axial pressure is applied to the first and second tip members of the removable tip configured as cross-linking tweezers, the distal ends of the first and second tip members are separated by a distance from one another in an open position and when axial pressure is not applied to the first and second tip members of the removable tip configured as cross-linking tweezers, the distal ends of the first and second tip members move proximate each other in a closed position;

Element 3: where the removable tip is configured as standard tweezers;

Element 4: when axial pressure is applied to the first and second tip members of the removable tip configured as standard tweezers, the distal ends of the first and second tip members move proximate each other in a closed position and when axial pressure is not applied to the first and second tip members of the removable tip configured as standard tweezers, the distal ends of the first and second tip members of the removable tip configured as standard tweezers are separated by a distance in an open position;

Element 5: where the removable tip configured as standard tweezers further comprises a locking mechanism coupled to the first and second tip members for locking the removable tip in the closed position;

Element 6: where the removable tip configured as standard tweezers further comprises a locking pin positioned in a lateral slot of the locking mechanism;

Element 7: where the locking mechanism of the removable tip configured as standard tweezers is moved toward the distal ends of the first and second tip members when the distal ends of the first and second tip members are in the closed position and the locking pin is engaged in the lateral slot to maintain the locking mechanism in a locked position;

Element 8: where the distal end of the first tip member is removable at or distal to a distal end of the first separating member and the distal end of the second tip member is removable at or distal to a distal end of the second separating portion;

Element 9: where the distal ends of the removable first and second tip members couple with the first and second separating members by a snap fit;

Element 10: where the first and second tip members each are a single piece from their distal to proximal ends;

Element 11: where at least the distal ends include a coating on at least an outer surface thereof;

Element 12: where at least the distal ends of the first and second tip members comprise at least one electrically conductive surface;

Element 13: where smoke cannot escape between either an inner or outer surface of the manifold and either an outer or inner surface of the shroud;

Element 14: where the distal ends of the removable first and second tip members couple with the first and second separating members by a tension fit or a locking fit; and

Element 15: where at least the distal ends of the first and second tip members include a non-stick coating.

Those skilled in the art to which this application relates will appreciate that other and further additions, deletions, substitutions and modifications may be made to the described embodiments.

What is claimed:

1. A removable tip for use with an electrosurgical tool, comprising:
a first tip member having a proximal end, a distal end, and a first separating portion located therebetween;
a second tip member having a proximal end, a distal end, and a second separating portion located therebetween, wherein the proximal end of the second tip member is directly coupled with the proximal end of the first tip member;
a smoke collector comprising at least one extension tube and a manifold; and
an insertion member directedly coupled with the proximal ends of the first and second tip members, the insertion member for inserting the removable tip within an opening in the electrosurgical tool; wherein:
the at least one extension tube is affixed to an edge of the first or second separating portions, the at least one extension tube having a first opening and a second opening, the first opening at a first end of the at least one extension tube closest to the distal ends of the first and second tip members and the second opening at a second end of the at least one extension tube opposite the first end of the at least one extension tube;
each of the second ends of the at least one extension tube is sealingly attached to the manifold;
the at least one extension tube configured to allow smoke to be evacuated away from an area at the distal ends of the first and second tip members through the first end of the at least one extension tube into the manifold through the second end of the at least one extension tube.

2. The removable tip according to claim 1, wherein the removable tip is configured as cross-linking tweezers.

3. The removable tip according to claim 2, wherein:
when axial pressure is applied to the first and second tip members, the distal ends of the first and second tip members are separated by a distance from one another in an open position; and
when axial pressure is not applied to the first and second tip members, the distal ends of the first and second tip members move proximate each other in a closed position.

4. The removable tip according to claim 1, wherein the removable tip is configured as standard tweezers.

5. The removable tip according to claim 4, wherein:
when axial pressure is applied to the first and second tip members, the distal ends of the first and second tip members move proximate each other in a closed position; and
when axial pressure is not applied to the first and second tip member, the distal ends of the first and second tip members are separated by a distance in an open position.

6. The removable tip according to claim 5, further comprising a locking mechanism coupled to the first and second tip members for locking the removable tip in the closed position.

7. The removable tip according to claim 6, further comprising a locking pin positioned in a lateral slot of the locking mechanism.

8. The removable tip according to claim 7, wherein:
the locking mechanism is moved toward the distal ends of the first and second tip members when the distal ends of the first and second tip members are in the closed position; and
the locking pin is engaged in the lateral slot to maintain the locking mechanism in a locked position.

9. The removable tip according to claim 1, wherein the distal end of the first tip member is removable at or distal to a distal end of the first separating portion and the distal end of the second tip member is removable at or distal to a distal end of the second separating portion.

10. The removable tip according to claim 9, wherein the distal ends of the first and second tip members couple with the first and second separating members by a snap fit.

11. The removable tip according to claim 1, wherein the first and second tip members each are a single piece from their distal to proximal ends.

12. The removable tip according to claim 1, wherein the first and second tip members include a coating on at least an outer surface thereof.

13. The removable time according to claim 1, wherein at least the distal ends of the first and second tip members comprise at least one electrically conductive surface.

14. An electrosurgical tool, the electrosurgical tool comprising:
a handheld electrosurgical unit, the handheld electrosurgical unit configured to be connected with an electrosurgical wall unit and including:
at least a first button to activate a cutting mode;
a second button to activate a cauterizing mode; and
a shroud configured to evacuate smoke through the electrosurgical tool; and
a removable tip; the removable tip comprising:
a first tip member having a proximal end, a distal end, and a first separating portion located therebetween;
a second tip member having a proximal end, a distal end, and a second separating portion located therebetween, wherein the proximal end of the second tip member is directly coupled with the proximal end of the first tip member;
a smoke collector comprising at least one extension tube and a manifold; and
an insertion member directedly coupled with the proximal ends of the first and second tip members, the insertion member for inserting the removable tip within an opening in the electrosurgical tool; wherein:
the at least one extension tube is affixed to an edge of the first or second separating portions, the at least one extension tube having a first opening and a second opening, the first opening at a first end of the at least one extension tube closest to the distal ends of the first and second tip members and the second opening at a second end of the at least one extension tube opposite the first end of the at least one extension tube;
each of the second ends of the at least one extension tube is sealingly attached to the manifold;
the at least one extension tube configured to allow smoke to be evacuated away from an area at the distal ends of the first and second tip members through the first end of the at least one extension tube into the manifold through the second end of the at least one extension tube to the shroud of the electrosurgical device.

15. The electrosurgical tool according to claim 14, wherein smoke cannot escape between either an inner or outer surface of the manifold and either an outer or inner surface of the shroud.

16. The electrosurgical tool according to claim 14, wherein the removable tip is configured as cross-linking tweezers.

17. The electrosurgical tool according to claim 14, wherein the removable tip is configured as standard tweezers.

18. The electrosurgical tool according to claim 14, wherein the distal end of the first tip member is removable at or distal to a distal end of the first separating portion and the distal end of the second tip member is removable at or distal to a distal end of the second separating portion.

19. The electrosurgical tool according to claim 18, wherein the distal ends of the first and second tip members couple with the first and second separating members by a tension fit or a locking fit.

20. The electrosurgical tool according to claim 14, wherein at least the distal ends of the first and second tip members include a non-stick coating.
